(19) United States
(12) Patent Application Publication
(10) Pub. No.: US 2010/0191263 A1
(43) Pub. Date: Jul. 29, 2010

(54) PROSTHESIS FOR LAPAROSCOPIC ANASTOMOSIS

(76) Inventor: Luiz Gonzaga Granja Filho, Recife/PE (BR)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/303,827

(22) PCT Filed: Jun. 6, 2007
(86) PCT No.: PCT/BR07/00150

§ 371 Date: Apr. 6, 2010

(30) Foreign Application Priority Data
Jun. 6, 2006 (BR) PI 0603438-1

(51) Int. Cl. A61B 17/11

(52) U.S. Cl. 606/153

(57) ABSTRACT

Prosthetic devices are provided for laparoscopic anastomosis, of end-to-side, end-to-end and side-to-side, with clamping and sutureless or with quick clamping and sutureless, where a prosthesis of large caliber can be introduced in the inner of the cavity where the anastomosis will be done passing through trocars of small caliber. When the prosthesis has intraluminal part, this will be covered by the grafts avoiding the contact of fluids which flow with the prosthesis material and with the anastomosis threads. The flange or the intraluminal part can also have openings or small straps allowing the fixation of the prosthesis to the external part of the tissue, vein, artery or tubular organ to eliminate the contact of foreign bodies with the inner of the anastomosis.

### INVENTION FIELD

The present invention refers, in general, to anastomotic devices and, more specifically, to a prothetic device which allows the confection of anastomosis without clamping and sutureless by laparoscopic route, where at least one vascular graft, or any other graft, is inserted in the prosthesis lumen or is circumferentially involved by it, and is turned by jacketing to cover part of the prosthesis, which will remain inside the graft (vein, artery or any other tubular organ) and is fixed to the tubular member or prosthesis flange by circular or single points. The flange or tubular member of the prosthesis can have a plurality of spaced openings on its margin or edge, or dents in the perimeter, allowing the prosthesis to be sutured outside the anastomosis, in the tissue, vein, artery, or any other organ, thus eliminating one of the main causes of anastomosis obstruction which is the introduction of foreign bodies inside its lumen.

### DESCRIPTION OF THE PRIOR ART

A prior art presents several trials provide solutions for anastomotic devices projected to correct vascular abnormalities, which present the following typical features:

The North-American U.S. Pat. No. 3,254,650, of Jun. 7, 1966, describes a method and devices to execute anastomosis procedures by applying with adhesive two separated connectors in a body member and removing this body member portion contained among the connectors, joining the said connection devices for joining the remaining portions of the body member.

The U.S. Pat. No. 3,265,069, of Aug. 9, 1966, describes devices or instruments for use by surgeons in reunion of body ducts, which in the course of operations were separated. The instruments comprise a pair of elongated similar elements and articulatedly connected, in an intermediary manner, and with an support for finger retention in a distal end, comprising a generally cylindrical shape with a cylindrical channel that passes through it in the other distal end, in order to receive tubular body ducts kept by the instrument while the body ducts are reconnected.

U.S. Pat. No. 3,774,615, of Nov. 27, 1973, describes a device to connect the end of interrupted tubular organs without sewing, comprising a connecting ring on which the end of the interrupted organ are pulled, the ring is preferably locked up by a fixation resource. The ring and fixation resource are made of inert material, and preferably a hydrophile gel that can be dilated until its equilibrium or can be a hydrogel incompletely dilated, which is submitted to additional dilatation where it is applied. The connecting ring can be supplied with a groove and can be placed in a ring shaped fixation resource and kept there joining it to the fixation resource in the groove or simply kept by a screw. Two connection rings can also be used and kept joined by a coupling member.

The document U.S. Pat. No. 4,366,819, of Jan. 4, 1983, describes an anastomotic joint for surgery with a graft of coronary artery deviance comprising a mounting of four elements including a cylindrical tube with at least one locking indentation of ring flange in one influx end and a plurality of grooves of locking ring in a flow end; a ring flange with a central opening and a plurality of long and short spigots, the long spigots are engaged in the locking indentation, with a graft engaged among them; a fixation ring with a central opening and a plurality of spigots positioned around the opening; and a locking ring with a opening with a plurality of locking ring edges for engaging with the locking ring grooves. In surgical implants, an aortic wall with a hole engages between the ring flange and the fixation ring and is kept in this position by spigots of the fixation ring, and the four elements engage together forming an integral anastomotic joint. A first alternate modality includes an anastomotic joint of three elements with a combination of fixation ring and locking ring. A second alternate modality includes an anastomotic joint of four elements with a slightly jolted end in a influx end, exposing the graft material in the anastomotic “ostium”.

Other prior arts are equally mentioned, base don some information of “The Cardiothoracic Surgery Network”. The “Simmetry Aortic Connector System”, developed by St. Jude Medical, is a connector made with nitinol, selected by vein diameter with an adventitia removed to allow adjust of the connector and to prevent its displacement by the blood current. Then, the device may make an angle of 90° with the aorta. Among the disadvantages, there is the fact that it can be used only in extreme cases due to the difficult usage of this technique; it did not obtain a satisfactory result in many surgeries and it is being drowned out of market by the manufacturer; it is not applicable in calcified aorta; presents suture; presents contact with blood flow (foreign body); it does not widen the anastomosis area (restrictive anastomosis); performs only one anastomosis at a time; it is a product restrict to end-to-side anastomosis; a great mobilization of the venous graft occurs, damaging it, and can eventually form thrombus; there is a risk of perforation of the posterior wall of aorta; and the adventitia is removed (most resistant vascular layer).

Other known device is the PAS-Port™System, a device used in 3 steps, and the vein wall is mounted over the device and is manually reversed on it, by tool and adapted to aorta with a angle of 90°. The method alerts that the surgeon shall select with due care the point of aorta and the vein size. The device is made of stainless steel and is available in only one size that allows the use of veins with external diameter of 4 to 6 mm, aorta with an internal diameter of 18 mm. It is available in only one size, limiting its applicability. As disadvantages of this prior art, the device has contact with blood flow (foreign body); it does not widen the anastomosis area (restrictive anastomosis); it uses veins with external diameter of 4 to 6 mm and aorta with an internal diameter of 18 mm; it does not perform multiple nor visceral anastomosis; it performs just only end-to-side anastomosis; a great mobilization of the used biological graft occurs, damaging its inner layer, which generates the formation of thrombus; there is a big risk of kinking at the origin (angle of 90°) and risk of posterior wall perforation in the aorta at the moment the device is introduced under its light; the suture is substituted with disadvantages by stainless steel (9 pins, distant among them, maximizing the risk of bleeding).

Also as prior art, there is the CorLink Device, currently commercialized by Ethicon/Johnson & Johnson, that allows the creation of anastomosis between the ascending aorta and a saphenous vein segment. Aortic Anastomotic Device (AAD) is a self-expanded device with extra luminal nitinol constituted by a de um central cylinder with five interconnected elliptical arches and 2 groups of 5 pins in the end portion of the cylinder. The pins, after the eversion of venous walls in the device, fix the aggregate penetrating into the venous graft wall. A blade makes an opening in the wall of aorta and permits the coupling of AAD, which also fix the wall of aorta by pins. With this device: it poses a serious risk of bleeding, especially in friable aortas, thin, calcified or fibrous, restricting its applicability, also with risks, even in aortas with normal walls; in small gauge anastomosis, there is a risk of thrombosis, hyperplasia, intimal proliferation and fibrosis (reaction to foreign body type in origin of anastomosis) with consequent stenosis resulting in occlusion of anastomosis; sutures are used in some cases; there is cases of infarction caused by equipment; there is a recurring need of re-operations in patients; the device presents contact with blood flow (foreign body); it is not flexible; it does not multiple anastomosis; an inadequate mobilization of venous graft occurs, and can cause damage to its intimal layer, it could form thrombus; it is used only in extreme cases because it is a technique of complex usage; the suture is substituted by stainless steel in contact with blood flow.

Another known device is the St Jude Distal Connector that consists of a stainless steel clip mounted on a catheter, comprising a balloon for subsequent expansion and connector mounting. The catheter is introduced backward from the end, by doing a small hole in the anastomosis site, the clip fixes the vein in the hole, the catheter goes to coronary and releases the connector. The catheter is removed and a suture is done in side-to-side anastomosis. With St Jude Distal Connector, occurrence of leakage problems were detected in 20% of the used connectors; the use of a metallic clip requires due care for handling to avoid distortion in the anastomosis; late angiographies reveal smaller circular diameter of anastomosis made with o St. Jude Distal Connector, when compared to controls made with conventional suture; there is remarkably risk of bleeding and the graft is very mobilized, and lacerations can occur in its inner layer, allowing the formation of thrombus.

The HeartFlo™ is a multi-suture instrument for anastomosis with wires automatically applied in end-to-side and side-to-side anastomosis. The surgeon manually ties the suture wires (10 wires) and concludes the anastomosis similarly to the traditional process. Besides of being a product of complex handling, it makes suture in anastomosis (keeping the undesirable foreign body in the internal origin of the anastomosis) and is restricted to end-to-side and side-to-side anastomosis. There is also an excessive mobilization of graft, and can cause lesions in its intimal layer, which would be the inductor that forms the thrombus.

Another technique and known device is the Solem Graft connector, produced by the Swedish company Jomed. It is constituted by a stent made of nickel and titanium coated with polytetrafluorethylen used to connect the internal thoracic artery the left anterior descending coronary artery. The results has not been satisfactory, because it poses risk of bleeding; there is also an excessive mobilization of graft, probably damaging intimal layers, allowing the formation of thrombus; it is not flexible, by this fact, causes trauma to grafts; it does not make multiple anastomosis, at a single time; presents contact with blood flow (foreign body); and is frequent the need of-operations.

The Magnetic Vascular Positioner System is produced by Ventrica and comprises 4 magnetic rings and the anastomosis is processed by magnetic attraction of 4 ports. However, initial experimental results demonstrate leakage, also a undesired contact of materials with blood flow. On the other hand, it is necessary to be careful to avoid the capitation of excess of tissue among the magnets. With this system, there is also a need of suture in some cases; there is occurrence of infarction caused by equipment; and is frequent the need of-operations in patients; and also requires clamping.

Also, as a device known by the medical area, the Combined Anastomotic Device and Tissue Adhesive, developed by Grundeman & Borst group, combines micro mechanical technique with use of adhesive (glue). The use of this method can result in leakages and need traditional sutures; it is frequent the need of re-operation due to leakage/bleeding; and performs only one anastomosis at a time.

Finally, it is also experimentally practiced anastomosis assisted by laser, where the results are not different from conventional isolated sutures, because there is a need of suture in some cases; there is a risk of bleeding e leakage; and does not perform multiple anastomosis.

Even so divulged nowadays, anastomosis with damper, by insecurity, and almost totality of surgeons perform conventional sutures throughout the route of anastomosis, with an intention of avoiding leakages and bleedings, it means the use of dampers just makes the procedure more expensive, once the conventional suture is also applied.

In short, the conventional anastomosis, with clamping and with suture, standardized in 1906 by Aléxis Carrel, remains the first choice for any type of anastomosis and organs to be anastomosed.

With an expectation of changing the current situation, the Brazilian patent no. PI 9706197-2, describes and claims a prosthesis for vascular anastomosis, or in any other organ or tissue, without the use of clamping and sutureless, solving, in an elegant and efficient manner, the limitations inherent to prosthesis of the above mentioned prior art, when used in vascular anastomosis performed, mainly in thin aortas, calcified and friable; or in any other application where a clamping of a vein or artery can pose excessive trauma for conditions of a given patient. The prosthesis that is subject of that request allows the embodiment of fast and safe anastomosis, without obstruction of vein or artery lumen of which anastomosis is made, also allows anastomosis in tissues, veins or arteries in bad conditions and never would accept a clamping used in conventional anastomosis. This is achieved by a generally cylindrical shaped prosthesis with a flange orthogonally extending from its external side wall, in a point in the prosthesis length between its ends; the referred flange has openings distributed around its surface. The description of the usage method and specific construction of the prosthesis is presented in the drawings of the descriptive report of that request, as well as the document C19706197-2, Certificate of Addition of the first.

Although these anastomotic devices can be presented as suited to the purposes for which they were projected, they are not so suited for the purposes of the present invention, as described herein below.

### SUMMARY OF THE PRESENT INVENTION

The present invention refers to variations of the anastomotic devices currently known, in a way to allow side-to-side, end-to-end and end-to-side anastomosis by laparoscopic route without clamping and sutureless, or with quick clamping (if organs with normal walls or which clamping does not represent any risks) and sutureless, where at least one vascular graft, or any other graft, is inserted in the prosthesis lumen or is circumferentially involved by it, and turned by jacketing to cover part of the prosthesis, and is fixed to the tubular member or prosthesis flange, by circular or single points, or any other method. The prosthesis can be rigid, divided into articulated parts, which fit or slide between themselves, or flexible and malleable, in single part, with elastic memory, in order to allow its passage, independent of its external diameter, by small gauge trocaters to access the inner of cavities where the anastomosis will be confectioned. It can or cannot have flange, which can or cannot have openings, straps and dents, allowing it to be sewn in the external of the tissue, vein, artery or tubular organ to eliminate the contact of foreign bodies with the inner of the anastomosis. The prosthesis can also have various dimensions and shapeats to simultaneously accommodate sizes, numbers and various numbers of grafts.

A goal of the present invention is to provide an anastomotic device which allows by laparoscopic route the confection of any type, size or number or anastomosis without clamping and sutureless or, in organs of normal walls or on which the clamping does not represent risks, with quick clamping and sutureless.

Another goal of the present invention is to provide an anastomotic device, which allows the confection of anastomosis by laparoscopic route, which does not introduce any foreign bodies in touch with the inner layer of the grafts on the anastomosis site (anastomosis sutureless).

Another goal of the present invention is to provide an anastomotic device, which allows anastomosis by laparoscopic route without clamping and hermetically closed, with no possibility of anastomotic leakages.

Another goal of the present invention is to provide an anastomotic device, which allows the pershapeance of anastomosis by laparoscopic route on which the grafts do not hustle independently of the pressure to which they are submitted and to where they are being positioned.

Another goal of the present invention is to provide an anastomotic device, which allows by laparoscopic route single, broad and multiple anastomosis at one time.

Additional objectives of the present invention and other modalities will appear when the description continues. These modalities will be described in sufficient details to allow that field technicians implement the invention. Besides, it is understood that other modalities can be used and that structural changes can be pershapeed without missing the invention scope. In the attached drawings, similar reference characters designate the same parts or similar parts throughout all several views.

The following detailed description is not, however, to be taken into a limit sense, and the scope of the present invention is better defined by attached claims.

### BRIEF DESCRIPTION OF DRAWINGS

In a way that the invention can be more completely understood, it will be now described, by means of example, with reference to the attached drawings, of which:

FIG. 1 illustrates a view in perspective of a prosthesis for laparoscopic anastomosis, straight, flexible, with elastic memory, with pre-determined final shape (circular, elliptical, oval, etc.), containing flange with double trespassing orifices or straps in its inferior surface (not shown), with fitting extremities of several shapes as illustrated on FIGS. 1B and 1C, containing intraluminal part which has external grooves (not shown) for fixation of the everted grafts.

FIG. 1A illustrates a superior view of the prosthesis from FIG. 1.

FIGS. 1B and 1C illustrate some of the possible fitting ways of extremities of the FIG. 1 prosthesis.

FIG. 1D illustrates a superior view of the final shape assumed by the prosthesis from FIG. 1 immediately before the fitting of its extremities.

FIGS. 2-2H illustrate an anastomosis by laparoscopic route pershapeed in the inner of a cavity, with the prosthesis from FIG. 1.

FIG. 3 illustrates another concretion of prosthesis, flexible, with elastic memory, with pre-determined shape, which does not have intraluminal part, constituted by an elastic thread coated by biocompatible synthetic fabric flange (Teflon, PTFE, Dacron, Gor-tex) or biological (autologous—the elastic thread can be coated at the moment of surgery with, in example, a segment of saphenous vein—; homologous—dura mater, veins, pericardium, or human fasciae—; or heterologous—pericardium, veins, dura mater or fasciae from any animal, ex. Bovine and swine).

FIG. 3A illustrates a transversal cut of the prosthesis from FIG. 3.

FIG. 3B illustrates the prosthesis from FIG. 3 with final circular shape.

FIG. 3C illustrates the prosthesis from FIG. 3 with final elliptical shape.

FIG. 3D illustrates the prosthesis from FIG. 3 with the colored threads previously attached to it.

FIG. 3E illustrates the application of the prosthesis from FIGS. 3-3D in an anastomosis.

FIG. 4 illustrates the superior view of another concretion of prosthesis continuous, flexible, with elastic memory, with tissue flange, with no intraluminal part, with pre-determined final shape and a detail of its transversal cut.

FIGS. 4A-4C illustrate another concretion of presentation of the elastic ring from the prosthesis from FIG. 4.

FIG. 5 illustrates another concretion of prosthesis for laparoscopic anastomosis, with two rigid parts, non-flexible or elastic, of equal dimensions, with fittings and means of fixation by its external extremities, with resistant threads, of different colors, previously fixed to double orifices of its flange, with only one long thin needle which links the extremities of a single folded thread, forming two straps of the same color.

FIG. 5A illustrates a concretion of possible dimensions of some parts of the prosthesis from FIG. 5.

FIG. 5B illustrates a final superior view, with parts already fitted and fixated, of the prosthesis from FIG. 5.

FIGS. 5C and 5D illustrate possible fixation and fitting ways of the extremities of the parts of the prosthesis from FIG. 5.

FIG. 5E illustrates the separate parts of the prosthesis from FIG. 5, with the short single needle threads, long and thin, and double strap, previously tied to its flange, in the inner of the trocarter through which they will be introduced in the cavity where the anastomosis will be confectioned.

FIG. 5F illustrates the parts of the prosthesis from FIG. 5, before fitting, already in the inner of the cavity (abdomen, torax, etc.) where the anastomosis will be done.

FIG. 5G illustrates the prosthesis from FIG. 5 in the inner of the cavity where the anastomosis will be done, with the folded threads, shoer, forming double straps, resistant, with single needle, discreetly curve, long and thin, which links its extremities, previously fixed by binding or any method, to the double orifices of its flange, with its parts already fixed and fitted, and its hypothetical final diameter.

FIG. 6 illustrates another concretion of the prosthesis for anastomosis by laparoscopic route, divided into equal parts, semi-flexible, with elastic memory, deformable in the center of its halves, with fittings by their extremities, similar to the prosthesis from FIG. 5.

FIG. 6A illustrates some possible dimensions of some parts of the prosthesis from FIG. 6.

FIG. 6B illustrates the separate parts of the prosthesis from FIG. 6, deformed on their centers, allowing them through the trocarter which caliber is inferior to the half of the prosthesis.

FIG. 6C illustrates the prosthesis from FIG. 6 already in the inner of the cavity where the anastomosis will be done, with its parts already fitted and fixed.

FIG. 7 illustrates another concretion of the prosthesis for anastomosis by laparoscopic route, with flange and intraluminal part, divided into two separate parts, equal in shape and dimensions, with fitting through their extremities, in the inner of the trocarter which caliber is equal or bigger than the fourth part of the prosthesis diameter. Each half is divided in two smaller parts, rigid, non-flexible, linked by coil-type, folding-type or hinge-type device, etc. which allow the basculation one to another.

FIG. 7A illustrates some possible measures of some parts of the prosthesis from FIG. 7.

FIG. 7B illustrates a superior final view of the FIG. 7 prosthesis, with folded threads, short, forming double strap, resistant, with single needle, discreetly curve, long and thin, which links its extremities, previously fixed by binding or any method, to the double orifices of its flange.

FIG. 7C illustrates a view in perspective of one the separated halves of the prosthesis from FIG. 6, representing the two parts which constituted it united by folding or coil device, and the fitting and fixation means to the other separated half.

FIGS. 7D-7I illustrate a technical concretion of the utilization o the prosthesis from FIG. 7 in the confection of an end-to-end anastomosis.

FIG. 8 illustrates another concretion of the prosthesis for anastomosis by laparoscopic route, which differs from the prosthesis from FIG. 7 by having a bolt and not a coil or folding type device, to link both parts of each of the separated halves.

FIG. 8A illustrates some of the possible measures of some parts of the prosthesis from FIG. 8.

FIG. 8B illustrates a final superior view of the prosthesis from FIG. 8 featuring its final caliber and the different thread colors, folded, short, forming double strap of the same color, resistant, with single needle, discreetly curve, long and thin, which links its extremities, previously fixed by binding or any method to the flange.

FIG. 8C illustrates a perspective view of one of the halves of the prosthesis from FIG. 8 emphasizing the central bolt linking its two constituent parts.

FIG. 9 illustrates another concretion of the prosthesis for anastomosis by laparoscopic route, with flange containing double orifices or straps and tubular member, constituted by two halves, rigid or flexible, with elastic memory or not, kept united by hinge-type device, featured, emphasizing the maximum diameter of the prosthesis.

FIG. 9A illustrates the prosthesis from FIG. 9 with its halves linked by the fixation bolt, featured.

FIG. 9B illustrates the prosthesis from FIG. 9 with its opposed halves, linked between them by the fixation bolt, featuring the maximum height of the set, represented by the maximum diameter radius of the prosthesis.

FIG. 10 illustrates another concretion of the prosthesis for anastomosis by laparoscopic route constituted of two halves, which articulate through bolts in their extremities.

FIG. 10A illustrates the prosthesis from FIG. 10 with its juxtaposed halves basculating 180 degrees one related to another.

FIG. 10B illustrates another form of union of the two halves of the prosthesis from FIG. 10 with its halves with sequence extremities.

FIG. 10C illustrates the prosthesis from FIG. 10B with its superposed and crossed halves after the 180 degrees basculation one over the other.

FIG. 10D illustrates another form of linking the halves of the prosthesis from FIG. 10 which halves are of the same dimension and shape and are confronting through their extremities where they fit and fix between themselves.

FIG. 10E illustrates the prosthesis from FIG. 10D with its halves superposed and not crossed.

FIG. 10F illustrates a perspective view of the halves of the prosthesis from FIGS. 10-10E, with their double orifices next to their external edges.

FIG. 11 illustrates another concretion of the prosthesis for anastomosis by laparoscopic route composed by two halves which slide between themselves and are linked by clamps in opposed directions present in only one of the opposed extremities of each one of its halves, featuring the orifices existent on the extremities of each half, without clamp.

FIG. 11A illustrates the halves juxtaposed and fixed between them, of the prosthesis from FIG. 11.

FIG. 11B illustrates the superior view of the prosthesis from FIG. 11 after total sliding, between themselves, of its halves.

FIG. 12 illustrates another concretion of the prosthesis for anastomosis by laparoscopic route composed by three parts of equal dimensions and shapes, which fit and fix through bolts and basculate between themselves staying juxtaposed or forming circular prosthesis.

FIG. 12A illustrates the three constituent parts of the prosthesis from FIG. 12, their connections through the bolts, their double orifices, their external furrows, their small orifices on the free extremities of two of the parts, and the sliding movement between them.

FIG. 12B illustrates the prosthesis from FIG. 10 with its three juxtaposed parts, in the inner of a trocarter with a caliber much lower than the caliber of the prosthesis from FIG. 12.

### DESCRIPTION OF THE PREFERRED MODALITIES

Now with reference to the drawings, on which similar characters of reference denote similar elements through all views, the figures illustrate one of the means of performance of the present invention, in the form of a prosthesis for anastomosis by laparoscopic route constituted of at least one open or closed elastic body, containing or not flange and/or intraluminal part, of variable dimensions or constituted of multiple parts articulable among themselves.

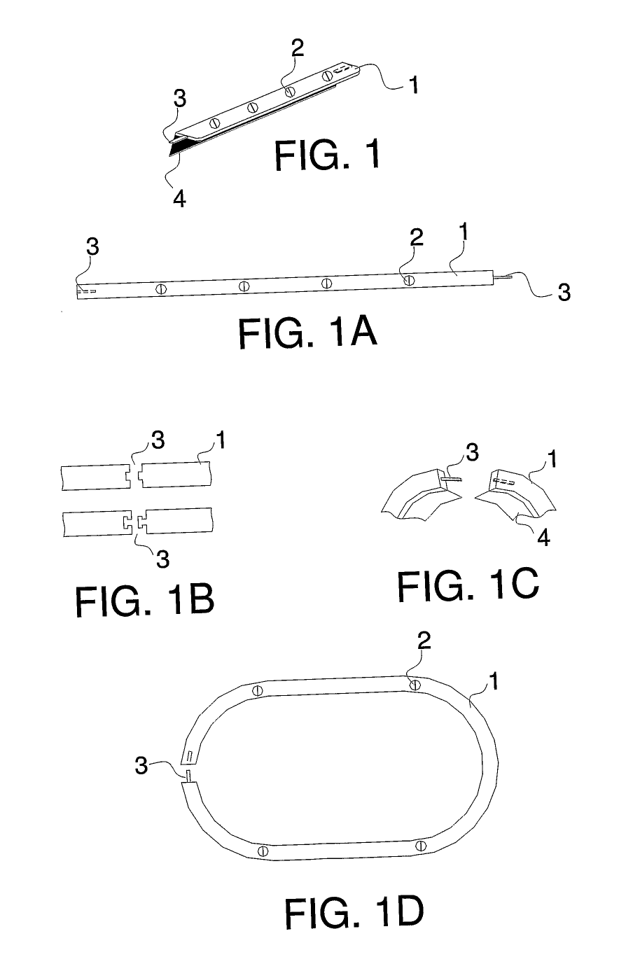

FIG. 1 illustrates a view in perspective of a prosthesis for laparoscopic anastomosis, straight, flexible, with elastic memory, with pre-determined final shape (circular, elliptical, oval, etc.), containing flange 1 with double trespassing orifices 2 or straps in its inferior surface (not shown), with fitting extremities 3 of several shapes as illustrated on FIGS. 1B and 1C, containing intraluminal part 4 which has external grooves (not shown) for fixation of the everted grafts 6,7. The fact that it is initially straight, gives it a small width and height allowing it to easily pass through trocaters 15 of thin caliber, for its introduction in the inner of the cavity where the anastomosis by laparoscopic route will be done, even if its final diameter, after fitting 3 of its extremities, is much bigger than the diameter of the used trocarter.

FIG. 1A illustrates a superior view of the prosthesis from FIG. 1, which will assume pre-defined shape when linking its extremities through its fitting 3 and fixation 14 forms.

FIG. 1B illustrates possible means of fitting between the opposed extremities of the same straight prosthesis. Countless concretions are possible.

FIG. 1C illustrates a perspective view of the flange 1, of intraluminal part 4, and the fitting 3 of the prosthesis from FIG. 1.

FIG. 1D illustrates a superior view of the final shape pre defined of a concretion of the prosthesis from FIG. 1, immediately before the fitting of its extremities.

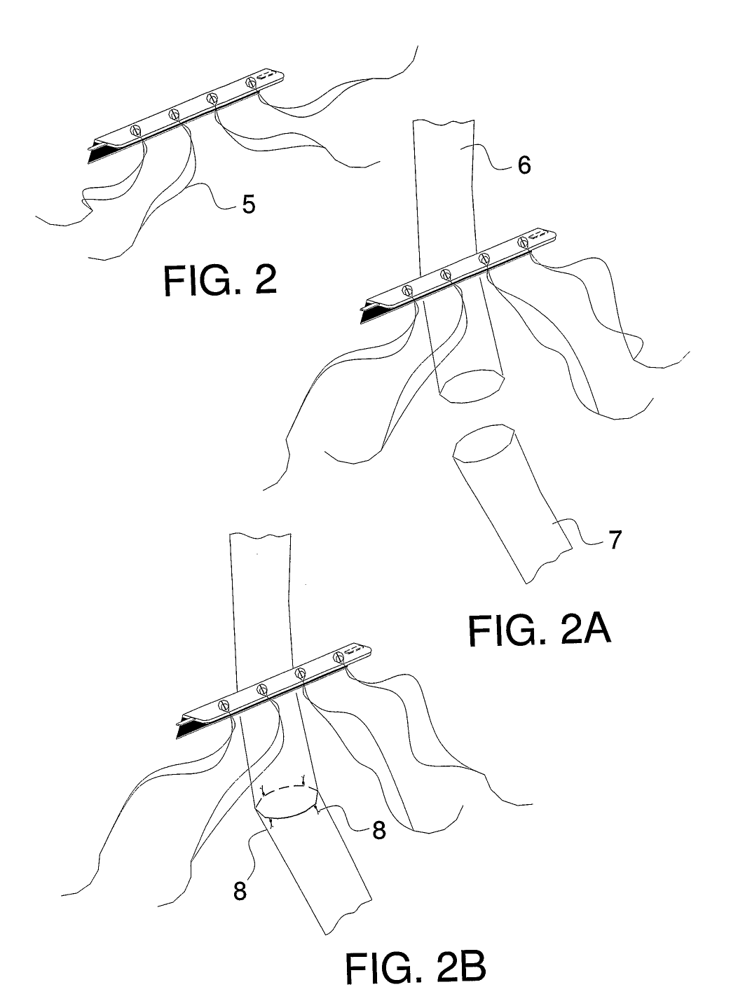

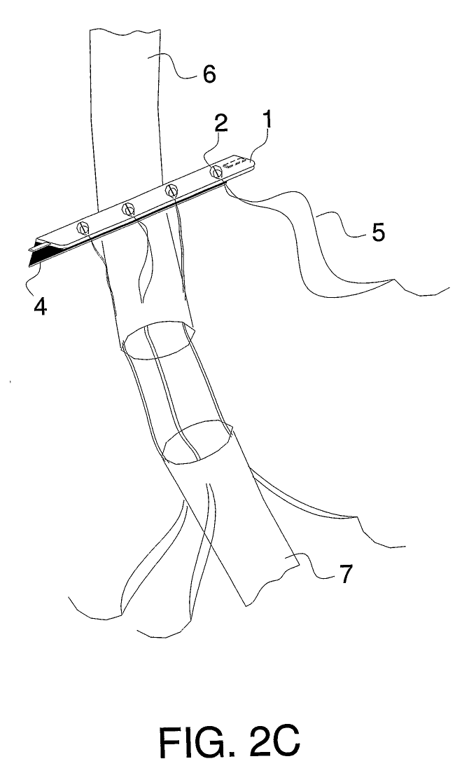

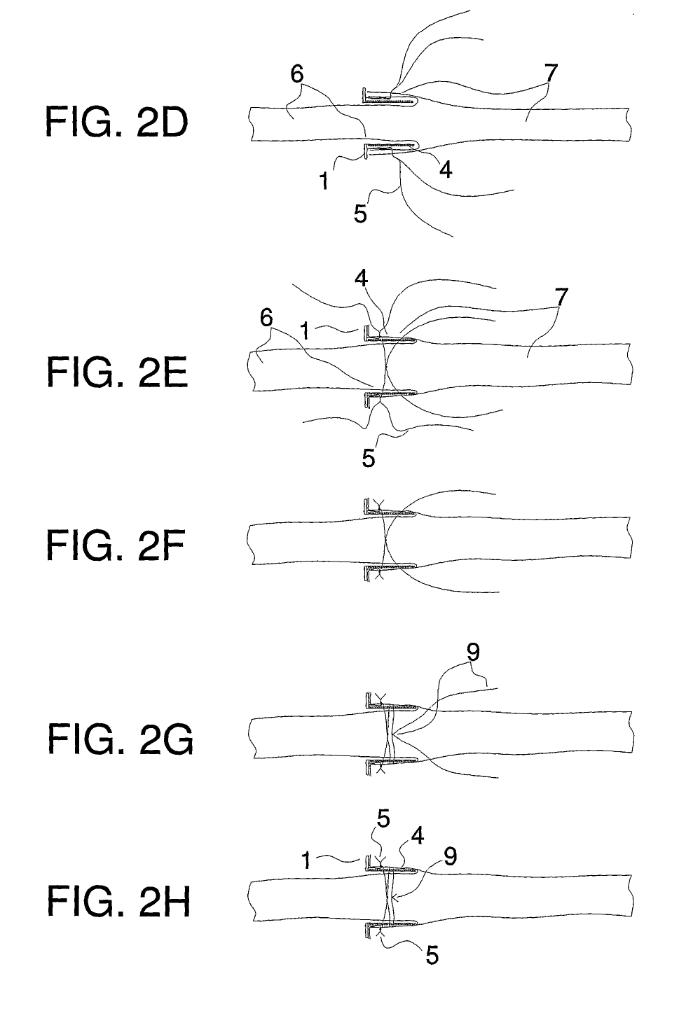

FIGS. 2-2H illustrate a technical concretion of the confection of an end-to-end anastomosis with the prosthesis from FIG. 1.

FIG. 2 illustrates the threads 5 of different colors interpolated, folded, short, forming double strap of the same color, resistant, with single needle, discreetly curved, long and thin, which links its extremities, previously fixed by binding or any method to the flange 1.

FIG. 2A illustrates the positioning of the prosthesis with the threads previously fixed to it, related to the grafts 6,7 to be anastomosed in end-to-end mode.

FIG. 2B illustrates the four simple points 8 which link the extremities of the grafts 6,7 aiming to facilitate the execution of the anastomosis procedure and covering of the intraluminal part 4 of the prosthesis by the extremities of the grafts 6,7. The application of these points 8, while recommendable, is not indispensable. Its applications 8 also facilitate, a lot, the visualization of the correct application sites for the anastomosis points 5. These simple points 8 could be replaced, on their functions, only by four little incisions, equidistant, longitudinal, situated on the extremity of the graft that will be everted. They equally would facilitate a lot its eversion and the correct application site for the points 5 of the anastomosis. They could also be used together: the four little incisions and the simple points 8 which would turn it even simpler.

FIG. 2C illustrates and features the route of the points 5 applied on the extremities of the grafts 6,7. This route is as follows: the two straps of the same thread 5 previously fixated to the double orifice 2 of the prosthesis, which individualize on the inferior surface of the flange 1, are passed contiguously, at the same time, linked only to a needle, from outside to inside, near the margin of the extremity of the graft 6 initially related to the prosthesis. The prosthesis can, as soon as it penetrates the cavity where the anastomosis is going to be performed, be united by extremities 3 rebuilding its pre-determined shape, or in the moment of the application of the points it can remain straight. Pass the prosthesis behind the graft 6, initially do not fix its extremities, apply the points and then fix the extremities of the prosthesis previously to the graft 6, seems to facilitate the procedure even more. The straps of the same points continue to transfix contiguously, at the same time, linked only to a needle, from the inside to the outside, the second graft 7, also close to the margin of its extremity. If the simple points 8 were applied previously linking the extremities of the grafts 6,7, those, on each two straps of the same thread 5, will be passed, at the same time, linked only to a needle, exactly on the half portion between two simple points 8 applied. In case the simple points 8 have not been applied, the anastomosis points 5 must be applied equidistant, equally dividing the perimeter of the extremities of the grafts 6,7. After the application of the four points 5 represented, their four needles will be removed and they will be tractioned and tied (two straps of different threads are tied, with different colors) generating the sequence of illustrated events illustrated on FIGS. 2D-2F. It is important to point that only two points could be applied resulting on the same security.

FIGS. 2D-2F illustrate the covering of the intraluminal part 4 of the prosthesis through the everted segment of the graft 6 and through the segment of the graft 7 which rides them, after the traction of the threads 5. An important detail to be considered is that mainly on organs with peristalsis, it must be always in the direction of the intraluminal part to the flange. This could prevent the occurrence of intussusception. In order to definitely avoid this risk, fix with the anastomosis points or simple points, externally or to the prosthesis, the stumps of the anastomosed organs.

FIG. 2G illustrates the double lace 9 around and externally to the anastomosis, over the intraluminal part 4 of the prosthesis, performed by the last thread 5 after its binding. This double lace guarantees that the anastomosis stays hermetically closed and also avoids any internal protuberance of the everted prosthesis-graft set 6, on the light of the other graft 7, however it is not indispensable, and it may not be performed.

FIG. 2H illustrates the final aspect of the end-to-end anastomosis with the straight, flexible prosthesis, by laparoscopic route, from FIG. 1. This anastomosis, besides having irreductible caliber, broad if desirable, does not have foreign bodies on its light, is hermetically closed, the grafts do not hustle, turns to be much less resistant than the own structure and wall of the grafts 6,7. In a form that when doing a great traction to undo the anastomosis, the graft walls 6,7 are ruptured, being not related to the anastomosis itself. On the conventional anastomosis, its area and site get extremely friable, compelling the surgical team to have extreme care on its mobilization so that laceration of the extremities of the grafts 6,7 and consequently anastomotic leakages will not occur, needing reinforcement points or even its reconfection.

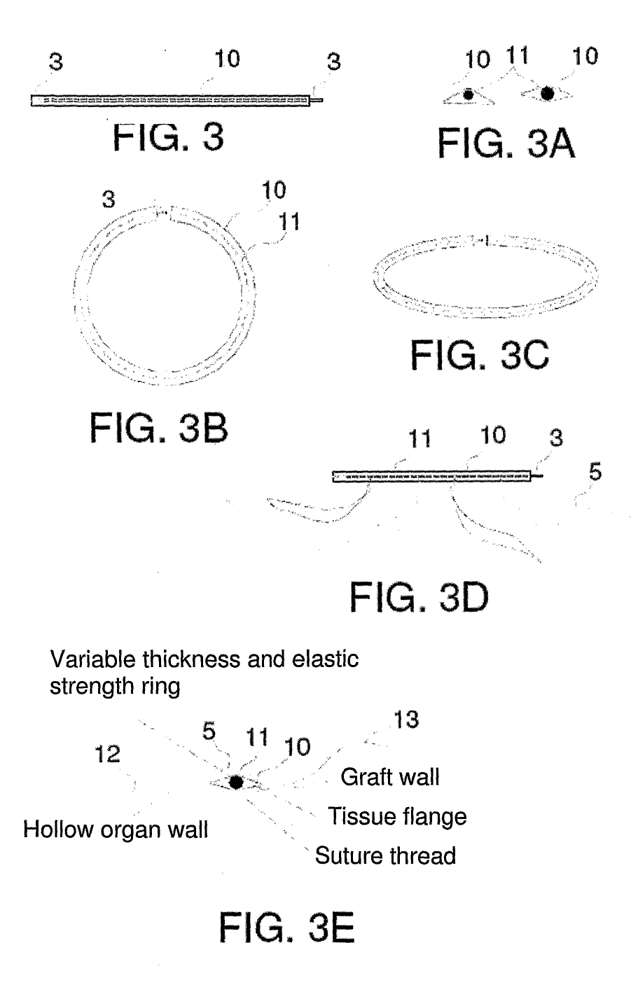

FIG. 3 illustrates another concretion of prosthesis which flange 10, is flexible, with no orifices, and biocompatible biological or synthetic fabric totally coats the flexible thread 11, of elastic memory, as illustrated in the transversal cut of FIG. 3 A. This prosthesis can or cannot have intraluminal part 4.

FIGS. 3B and 3C illustrate, respectively, final pre-determined forms, circular and oval, after fitting 3 of the extremities.

FIG. 3D illustrates the threads 5 of different interpolated colors, folded, short, forming double strap of the same color, resistant, with single needle, discreetly curved, long and thin, which links its extremities, previously fixed by binding or any method to the flange 10 of tissue, with no orifices, of the prosthesis from FIG. 3.

FIG. 3E illustrates a transversal cut of a end-to-end anastomosis with a concretion of a prosthesis with no intraluminal part 4 of the FIG. 3. The wall 12 of the organ, which receives the anastomotic set graft-graft 13 is noted. The threads 5 of different interpoled colors are evidenced, folded, short, forming double strap of the same color, resistant, with a single needle, discreetly curved, long and thin, which links its extremities, previously fixed by binding or any method to the flange 10, which transfixes twice the tissue flange 10 and has the following route: passes from the top to bottom in the tissue flange 10, outside to inside on the graft 13 which is being anastomosized, from inside to outside on the wall of the organ 12 which is receiving them, and again, from bottom to top, on the tissue flange 10 of the prosthesis, where they will be tied (straps of different colors of adjacent threads) after removal of their needles. In case the prosthesis has intraluminal part 4, the flange tissue 10 will not exist to the inside of the light of the prosthesis, and then the threads 5 of the anastomosis will only be fixed to it through their external part, similar to the threads 5 previously fixed on the prosthesis from FIG. 2.

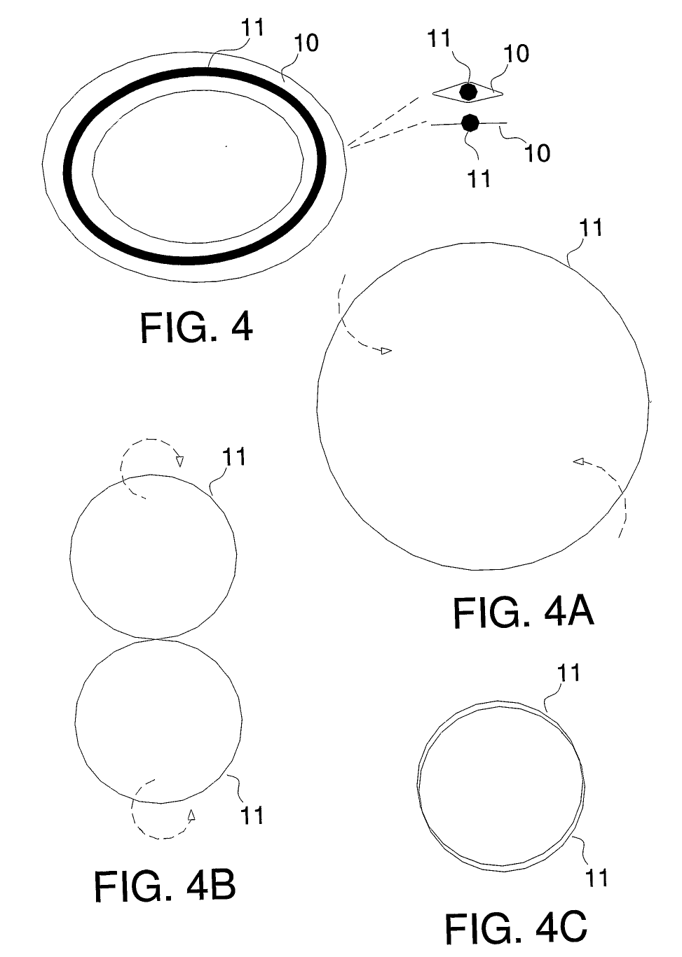

FIG. 4 illustrates another concretion of the prosthesis from FIG. 3, continuous, with tissue flange 10, flexible thread with elastic memory 11, which can be only compressed to decrease its diameter, or can be twisted over itself, as illustrated by FIG. 4A and FIG. 4B, forming initially, two halves with tangent extremities, continuous, crossed, being then folded one to another as illustrated on FIG. 4C, forming a circumference with diameter approximately equal to the half of its original size. This with the purpose of being able to pass through the light of trocarters 15 with caliber smaller than its original caliber, which will be reestablished as soon as it passes through the trocarter, already on the inner of the cavity where the anastomosis will be done. This prosthesis can or cannot have intraluminal part 4. In case of having intraluminal part 4, to facilitate its twisting over itself, on the site where it will be twisted, the intraluminal part 4, can be interrupted leaving only the elastic thread, which will also facilitate the folding over itself, forming the double circumference with the half of its original diameter, keeping, in this case, the intraluminal parts 4 in opposed directions.

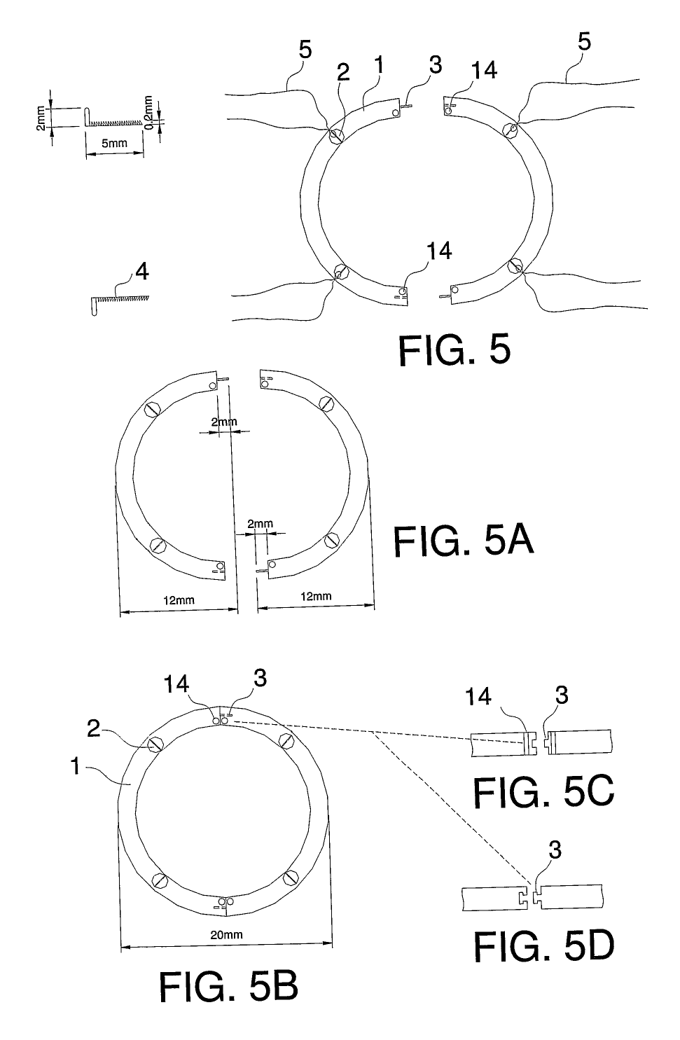

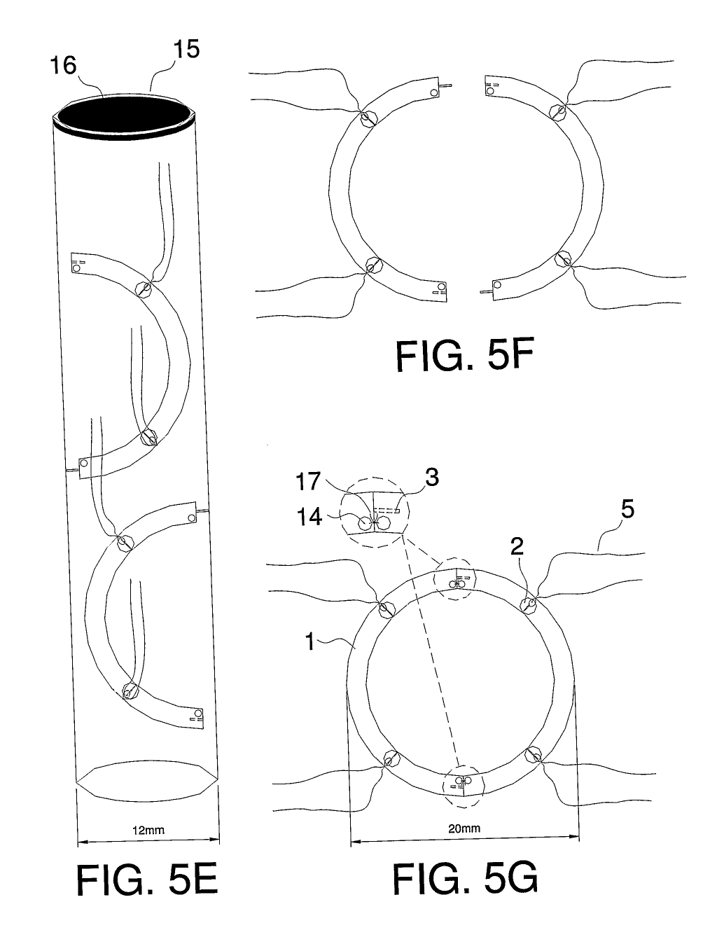

FIG. 5 illustrates another concretion of a prosthesis for anastomosis by laparoscopic route, which final dimensions are much bigger than the diameter of the trocarters 15 through which they will penetrate the cavity where the anastomosis will be done. This prosthesis is made of equal halves, rigid, with flange 4, double orifices 2, fittings 3, small orifices 14 on the edges of the flange extremities 1, through which points for reinforcement of the fixation of the halves of the prosthesis will pass. It is important to point that several other fitting concretions 3 and fixation 14 of the halves are possible.

FIGS. 5-5B illustrate possible dimensions of the prosthesis demonstrating that a prosthesis with 20 mm of final diameter, can pass through a trocarter 15 measuring only 12 mm of diameter, since its halves have maximum dimension of 12 mm.

FIGS. 5C-5D illustrate some fitting means 3 and fixation means 14 of the halves of the prosthesis. The mean illustrated on FIG. 5C will be safer if added to the small fixation orifices 14 through which simple points will pass for better fixation of the halves of the prosthesis. The mean illustrated on FIG. 5D can dispense the small orifices 14.

FIG. 5E illustrates the 12 mm halves of the prosthesis from FIG. 5, with the threads 5 of different interpolated colors, folded, short, forming double strap of the same color, resistant, with single needle, discreetly curved, long and thin, which links its extremities, previously fixed by binding or any method to the flange 1, in the inner of the trocarter 15, which has a diameter also of 12 mm. The trocarter 15 has elastic device 16 for contention of gases on its external extremity.

FIG. 5F illustrates the halves already on the inner of the organ where the anastomosis will be done following the linking of the halves through their fittings 3 and the small fixation orifices 14 through which they passed and the simple points 17 were binded to reinforce the fixation, as seen on FIG. 5G, rebuilding its original 20 mm caliber.

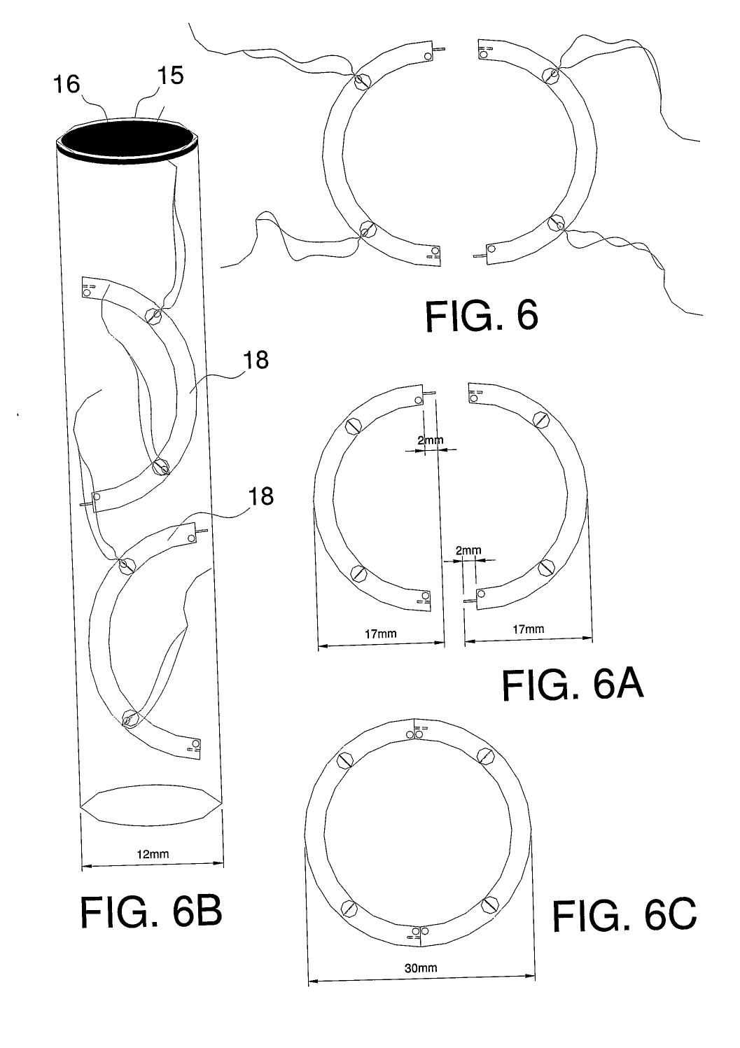

FIG. 6 illustrates another concretion of the prosthesis for laparoscopic anastomosis, which constitutent material is semiflexible, with elastic memory, allowing deformation 18 on the middle part of its halves, which allows them to enter the inner of a trocarter 15 of a much smaller caliber, 12 mm, than the biggest dimension of one of its halves, 17 mm, as seen on FIG. 6B. In the inner of the trocarter 15 occurs a stretching of its halves in its middle part, decreasing its dimensions for better accommodation to the smaller diameter of the trocarter 15.

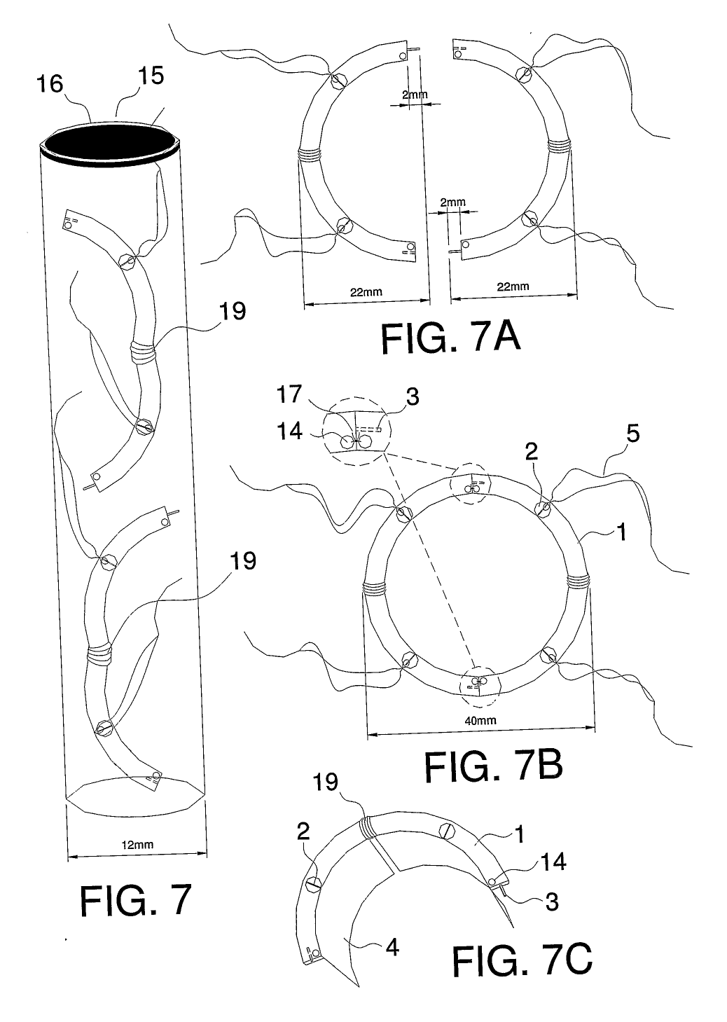

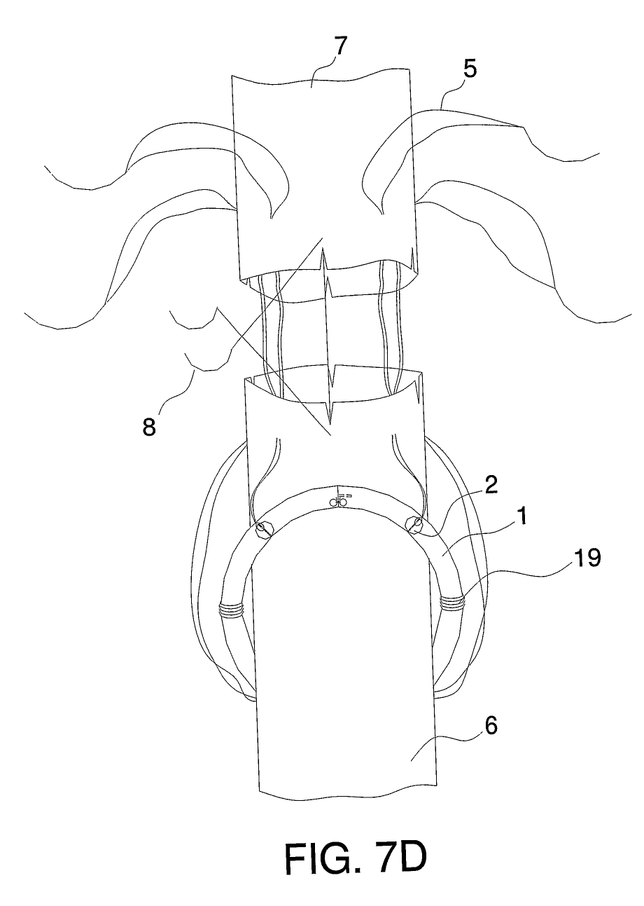

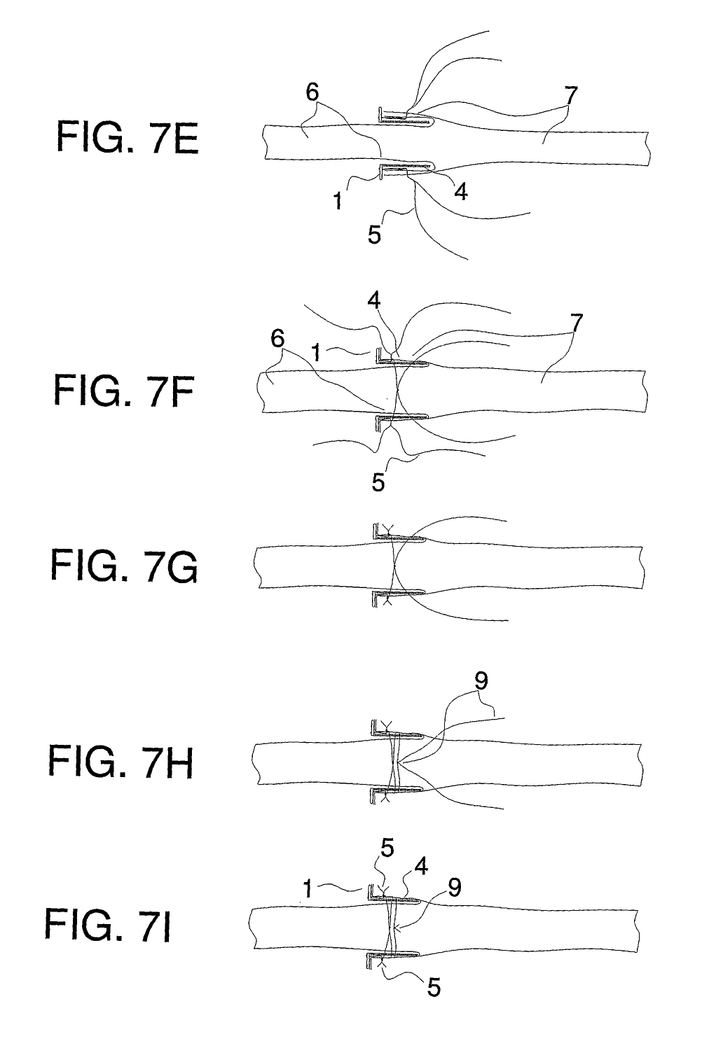

FIG. 7 illustrates another concretion of the prosthesis for laparoscopic anastomosis on which the middle part of its halves has an elastic device coil or folding-type 19, or any other device, which allows the total deflection of the middle part of the halves making each one with a maximum dimension equal to the half of its original dimension or ¼ of the dimension of the prosthesis. This aims to allow the passage of the prosthesis, of big final caliber, 40 mm, through the inner of a trocarter of a much smaller caliber, 12 mm, as illustrated on FIGS. 7-7B.

FIG. 7C illustrates a perspective view of one of the halves of the prosthesis from FIG. 7, where the elastic device coil or folding type 19 at flange 1 can be seen, and exactly over it 19, the interruption of the intraluminal part 4, discontinuous, which will allow the deflection for the inside of the middle part of the halves of the prosthesis. The double orifices of the flange 2, the fitting means 3 and the small fixation orifices 14 can also be seen.

FIG. 7D illustrates a technical concretion, already described, of the utilization of the prosthesis from FIG. 7 in a end-to-end anastomosis, which in this case had its halves previously fitted and fixed by simple points 17. Here the simple point 8 applied to previously link the extremities of grafts 6,7 can be seen, in order to facilitate the anastomosis procedure and eversion of the extremity of the first graft 6 for covering of the intraluminal part 4 of the prosthesis, which will be simultaneously covered by the non-everted extremity of the other graft 7, as the threads 5 of different interpolated colors, folded, short, forming double strap of the same color, resistant, with single needle, discreetly curved, long and thin, which links its extremities, previously fixed by binding or any method to the flange 1, are tractioned and tied as represented on FIGS. 7E-7G.

FIGS. 7H-7I illustrate, respectively, the double lace 9 of the last thread 5 over the prosthesis-grafts set 6,7, and the final aspect of the end-to-end anastomosis. As said, the double lace 9 over the grafts 6,7 and the intraluminal part of the prosthesis guarantee the hermetic character of the anastomosis, as well as the absence of protuberance from the intraluminal part 4—everted graft 6 set which covers it, on the light of the other graft 7. However, it is not indispensable and it may not be used.

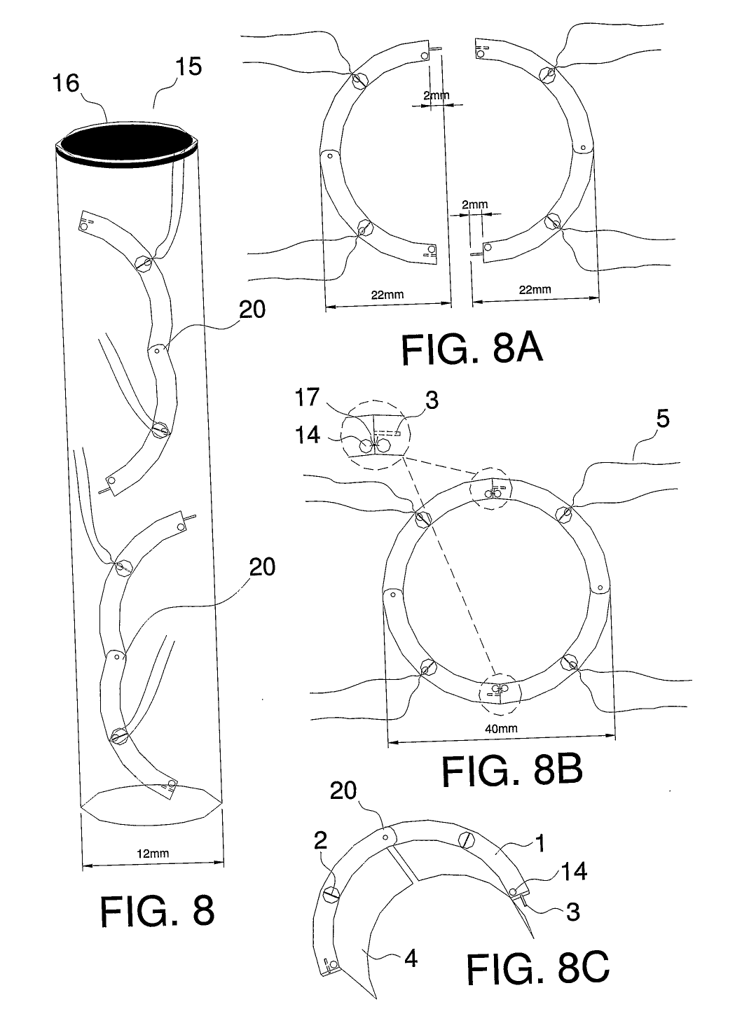

FIGS. 8-8C illustrate another concretion of prosthesis for laparoscopic anastomosis with the same characteristics of the prosthesis from FIG. 7, only differing for its linking mean 20 of the hemi-halves of the prosthesis. Here also occurs total deflection of the central part of the halves, making them of equal dimension of half of their original dimension, or ¼ of the maximum dimension of the prosthesis.

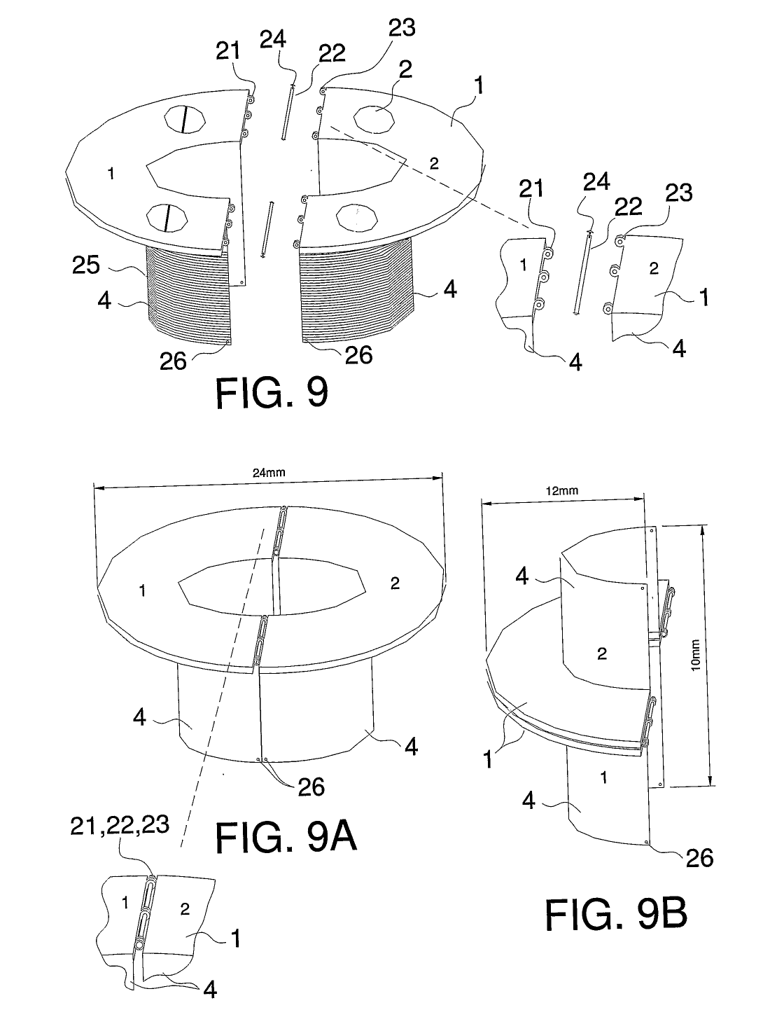

FIG. 9 illustrates another concretion of prosthesis for laparoscopic anastomosis, which principle is to allow that prosthesis of big caliber enter the cavities where the anastomosis will be performed, through trocarters of much smaller calibers. This is because the trocarters currently used have cylindrical form and maximum caliber of 12 mm, avoiding the confection of new trocarters of different dimensions and shapes (flattened, oval, rectangular, polygonal, etc.), as well as using minimum incisions for the introduction of prosthesis of big caliber.

The prosthesis illustrated on FIG. 9 has fitting and fixation device 21, 22, 23, 24, 26 of its halves and allow basculant movement of 180 degrees of one half related to the other as seen on FIG. 9B, reducing its maximum diameter to half. The central bolt 22 of the fitting device, has female thread in its external extremity where a small screw 24 will be placed, allowing its final fixation after passing by the orifices of the other fitting means and fixation means 21, 23. The external furrows 25 for circumferential fixation of the grafts to the intraluminal part are represented on FIG. 9, as well as the small orifices 26 or straps, situated near to the free edge of the intraluminal part, through which simple points 17 will pass for inferior fixation of the halves of the prosthesis. This way a prosthesis of 24 mm of maximum diameter can pass through a trocarter with 12 mm of diameter to be introduced in any cavity where the anastomosis will be performed.

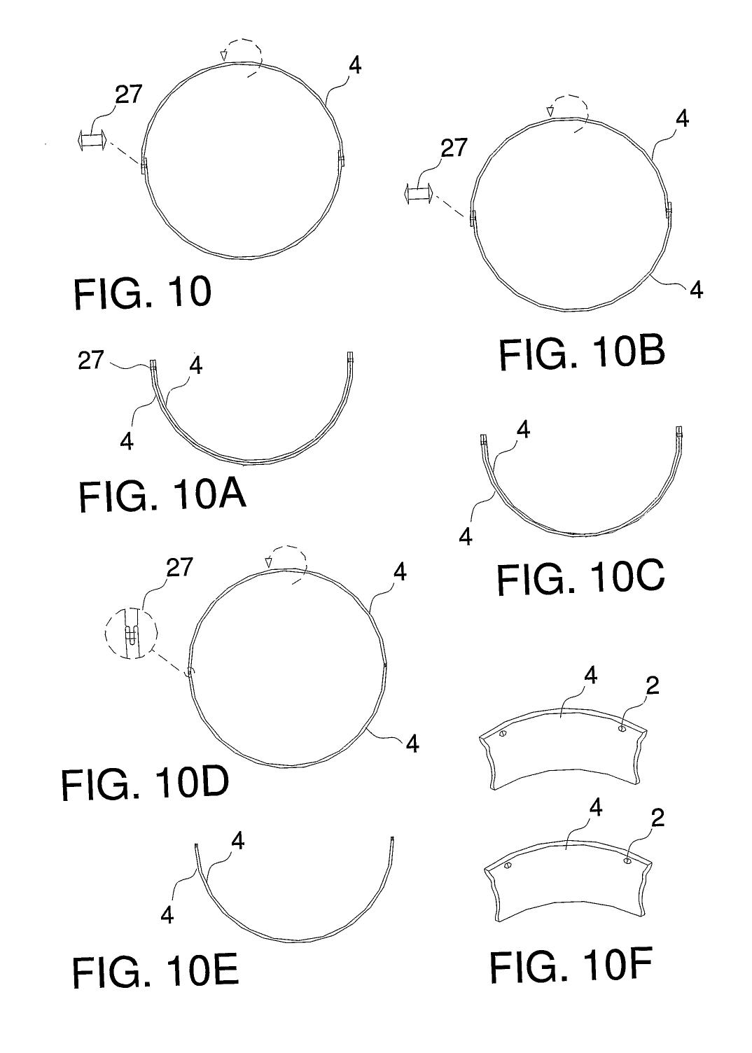

FIGS. 10-10E illustrate another concretion of prosthesis for laparoscopic anastomosis, without flange, which halves are articulated by bolts 27 which allow the basculation movement of 180 degrees of one half over the other, as the prosthesis from FIG. 9, however, without the flange 1, its halves sometimes juxtaposed, FIGS. 10-10A, sometimes crossed, FIGS. 10B-10C, sometimes superposed, FIGS. 10D-10E.

FIG. 10F illustrates a perspective view of fragments of the halves of the prosthesis from FIGS. 10-10E, on which the double orifices 2 are equidistant between themselves in the depth of the intraluminal part 4, not being represented the bolts 27 which articulate the halves.

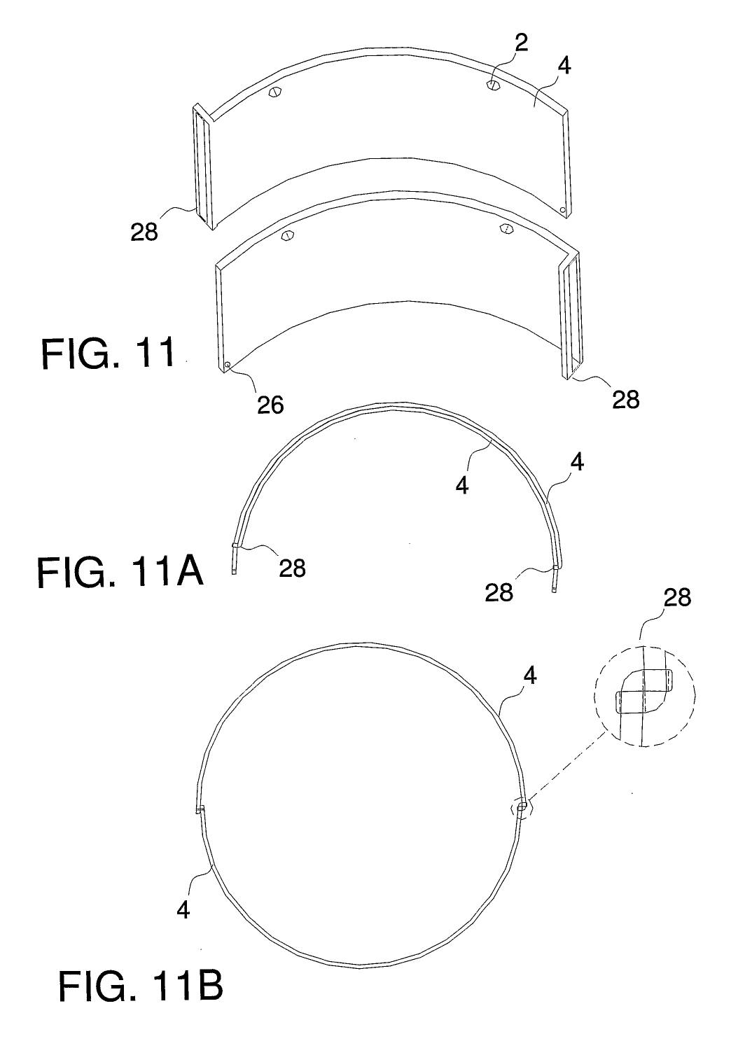

FIG. 11 illustrates another concretion of prosthesis for laparoscopic anastomosis, without flange 1, composed by two or more parts, which slide among them and fit through opposed straps 28, existent in one of the extremities of each part. Each part has on the inferior edge of its extremity without strap 28, small orifices 26, through which simple points will pass for stabilization and fixation of the parts.

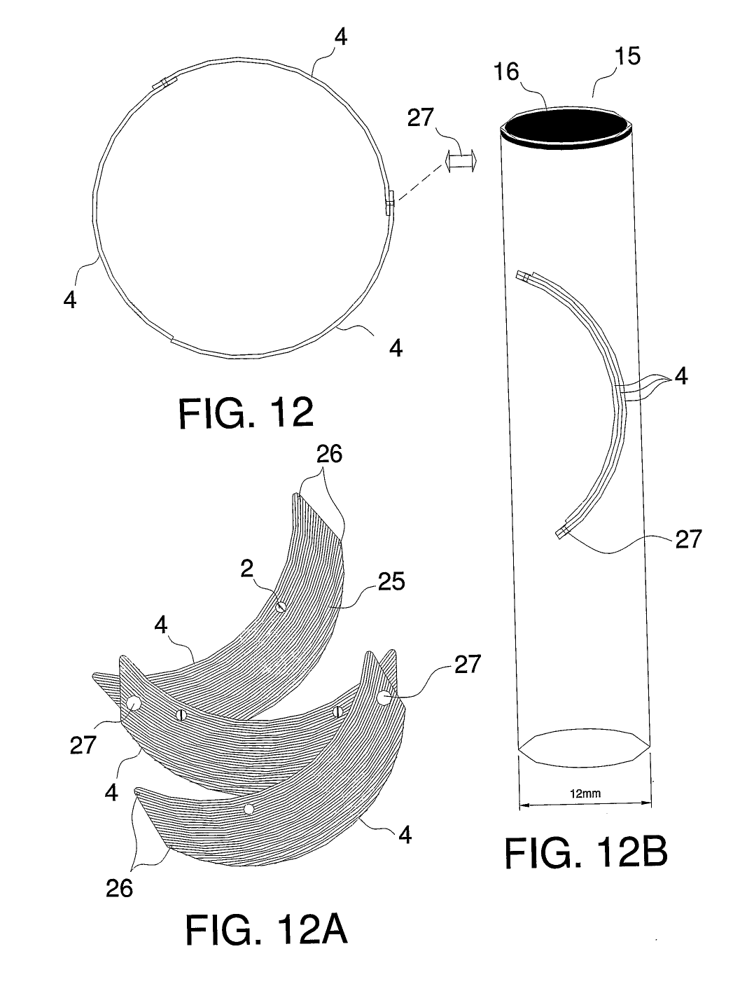

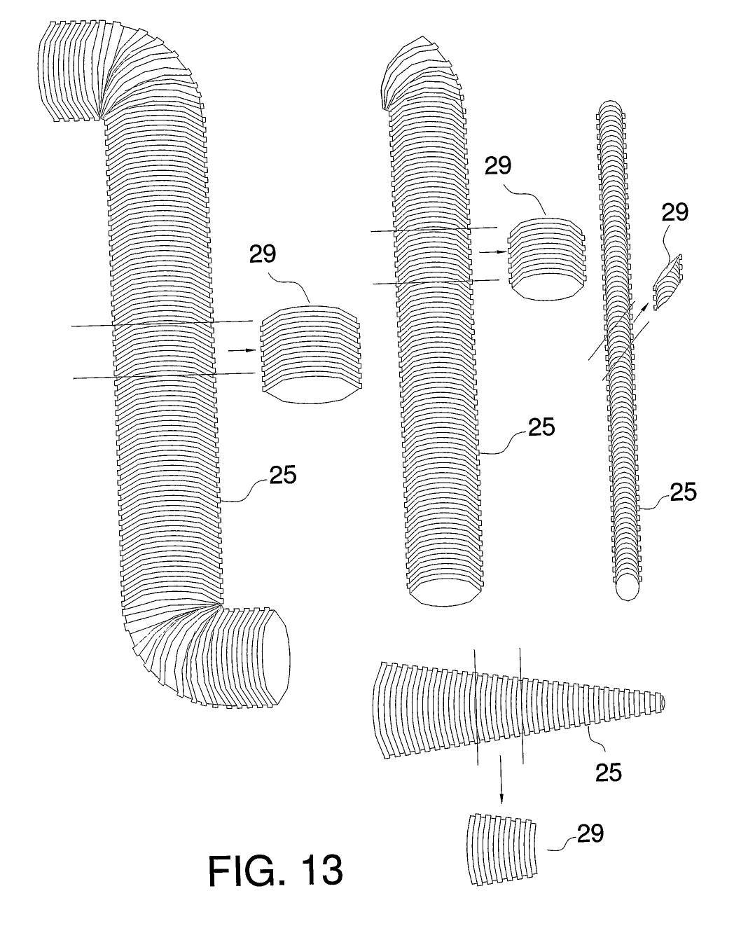

FIG. 12 illustrates a prosthesis for laparoscopic anastomosis, of final cylindrical form, which final or initial dimension is much bigger than the diameter of the trocarter 15 through that it will pass to enter the cavity where the anastomosis will be performed. It is composed by three parts articulated among themselves by bolts 27 which allow the rotative movement of 360 degrees of one part over the other, in a way that they are juxtaposed making a circumference arch with radium equal to ⅓ the radium of the prosthesis, as illustrated on FIGS. 12A-12B.

FIG. 12A illustrates the flanges 4, the double orifices 2, the external furrows 25 of the flange for circumferential fixation of the grafts, the small orifices 26 on the edges of the flanges, through where the simple points 17 for final fixation of its parts will pass, and the bolts 27 which articulate the parts among themselves.

Finally, FIG. 12B illustrates the prosthesis from FIG. 12, with its juxtaposed parts in the inner of a small diameter trocarter.

FIG. 13 illustrates a flexible tube, cylindrical, oval, elliptic or of any shape, silicon type or any other type, with elastic memory, biocompatible, of any caliber, resistant even with small depth of its walls, which can be sectioned transversally in any extension, being a transversal section, orthogonal, or in any angle, continuous or interrupted by longitudinal section of its tubular member 4, without or with single or multiple bevel, with one or more extensions of equal calibers, Medusa head-type, for anastomosis of several grafts with the same prosthesis. This tube has furrows 25 in straight angle in its external surface making it rugged, non-skidding, where the grafts will be fixed with circular points. Its internal surface is smooth. At the moment of surgery, depending on the caliber and depth of the wall of the organ to be anastomized, bigger or smaller extension and bigger or smaller caliber and shape could be confectioned. Each section 29 of the tube is now a prosthesis. The four threads can be previously applied, equidistant among themselves, transfixing its wall from inside the light to the outside, binding them over the external edge of the prosthesis. The anastomosis procedure is the one already described.

1. Prosthesis for laparoscopic anastomosis comprising: at least one flange (1), a lumen and an intraluminal part (4); the at least one flange (1) extending from a lateral wall of the intraluminal part (4); the at least one flange (1) and the intraluminal part having a plurality of through openings (2) distributed on a surface thereof, wherein the at least one flange, lumen and intraluminal part form a single, straight, flexible structure, with elastic memory, with fittings (3) and fixation member (14) through its extremities, with pre-determined final shape.

2. Prosthesis, according to claim 1, wherein the fitting (3) and fixation member (14) of the extremities are in the flange, and wherein threads (5) can be of different interpolated colors, folded, short, forming double strap of the same color, with single needle, discreetly curved, long and thin, linking its extremities, previously fixated by binding or any method to the through openings which can be double orifices (2) or straps on the flange (1) or in the intraluminal part (4).

3. Prosthesis, according to claim 1, wherein the intraluminal part can be covered by an everted graft and fixed to it pre-rounded or when the threads are tightened (5) to the anastomosis.

4. Prosthesis, according to claim 1, wherein the prosthesis assumes a pre-determined shape in an inner cavity where the anastomosis will be performed, by having elastic memory.

5. Prosthesis, according to claim 1, wherein the prosthesis can assume an open or closed configuration, as and when desired, so it can be introduced or removed from a cavity where the anastomosis will be performed, through a trocar (15) of a smaller caliber.

6. Prosthesis, according to claim 5, wherein the open configuration is a resting configuration and the closed configuration is taken on when it is to be adjusted to the smaller caliber of the trocar (15).

7. Prosthesis for anastomosis by laparoscopic route comprising: at least one, straight, open, flexible elastic body that extends from a flange, wherein the flange (10) comprises a flexible thread (11) having elastic memory and is made of fabric, flexible, with no orifices, that completely covers the flexible thread (11), with elastic memory.

8. Prosthesis, according to claim 7, further comprising an intraluminal part.

9. Prosthesis for anastomosis by laparoscopic route comprising: at least a tissue flange (10), without orifices, an elastic body (11), that is closed, circular, flexible, with elastic memory, with pre-determined shape, wherein the elastic body (11) can be twisted over a longitudinal axis and folded over itself.

10. Prosthesis for laparoscopic anastomosis comprising: at least one flange (1), an intraluminal part (4), double orifices (2), fitting member (3) and fixation member (14), threads (5) previously fixed to the at least one flange or intraluminal part, and at least two unelastic parts which are connected by their extremities.

11. Prosthesis, according to claim 10, wherein its parts have an intermediate semi-elastic part (18).

12. Prosthesis, according to claim 10, wherein its unelastic parts are further divided into two others linked through its flange or through its intraluminal part, through elastic device coil-type (19), folding-type (19), bolts (20), (27), screws (24), hinges (21), (22), (23) or any other fastener.

13. Prosthesis, according to claim 12, wherein its parts, in any number, can tilt, slide, spin, fold in any direction relative to one another.

14. Prosthesis for laparoscopic anastomosis comprising: a long, flexible, resistant, biocompatible tube, with elastic memory, of variable caliber and shape, without orifices or straps wherein the tube comprises furrows (25) in straight angle on an external surface thereof which is rugged and non-skidding.

15. Prosthesis, according to claim 14, wherein the elastic tube can be transversally sectioned, orthogonally or in any angle, in any extension and shape.

16. Prosthesis, according to claim 15, wherein a section (29) transversal, orthogonal or in any angle, of the elastic tube of variable dimensions and shapes, is a separate prosthesis.

17. Prosthesis, according to claim 16, wherein a section (29) transversal, orthogonal or in any angle, or new prosthesis, has, previously fixed to its tubular member, four threads double needled or with single needle.
